(19) United States
(12) Patent Application Publication
(10) Pub. No.: US 2014/0214081 A1
(43) Pub. Date: Jul. 31, 2014

(54) SPINAL IMPLANT SYSTEM AND METHOD

(71) Applicant: WARSAW ORTHOPEDIC, INC., Memphis, TN (US)

(72) Inventors: Frank J. Schwab, New York, NY (US); Alison G. Powers, Memphis, TN (US)

(73) Assignee: WARSAW ORTHOPEDIC, INC., Memphis, TN (US)

(21) Appl. No.: 13/756,052

(22) Filed: Jan. 31, 2013

(51) Int. Cl. A61B 17/70

(52) U.S. Cl. CPC A61B 17/7059
USPC 606/246

(57) ABSTRACT

A spinal implant includes a wall having a first surface and a second surface configured to engage tissue. The wall defines a first opening and a second opening configured for disposal of a bone fastener. The wall further defines a cavity disposed between the surfaces and in communication with the second opening. A biasing member is configured for disposal in the cavity and is engageable with the bone fastener to resist backout of the bone fastener from the second opening. Systems and methods are disclosed.

### TECHNICAL FIELD

The present disclosure generally relates to medical devices for the treatment of musculoskeletal disorders, and more particularly to a surgical system including an anterior spinal implant and a method for deformity correction.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. Correction treatments used for positioning and alignment may employ implants, such as vertebral rods, plates and fasteners, for stabilization of a treated section of a spine. This disclosure describes an improvement over these prior art technologies.

### SUMMARY

Accordingly, a surgical system and method for treatment of a spine disorder are provided. In one embodiment, in accordance with the principles of the present disclosure, a spinal implant is provided. The spinal implant comprises a wall having a first surface and a second surface configured to engage tissue. The wall defines a first opening and a second opening configured for disposal of a bone fastener. The wall further defines a cavity disposed between the surfaces and in communication with the second opening. A biasing member is configured for disposal in the cavity and is engageable with the bone fastener to resist backout of the bone fastener from the second opening.

### BRIEF DESCRIPTION OF THE DRAWINGS

The present disclosure will become more readily apparent from the specific description accompanied by the following drawings, in which:

FIG. 1 is a perspective view of components of one embodiment of a system in accordance with the principles of the present disclosure;

FIG. 2 is a break away, perspective view of the components shown in FIG. 1;

FIG. 3 is a cross-section view of the components shown in FIG. 2;

FIG. 4 is a break away, perspective view of the components shown in FIG. 1;

FIG. 5 is a break away, plan view of the components shown in FIG. 1;

FIG. 6 is a cut away view of the components shown in FIG. 5;

FIG. 7 is a plan view of components of one embodiment of a system in accordance with the principles of the present disclosure disposed with vertebrae;

FIG. 8 is a break away, plan view, in part cross section of components of one embodiment of a system in accordance with the principles of the present disclosure; and

FIG. 9 is a break away, plan view of the components shown in FIG. 8.

Like reference numerals indicate similar parts throughout the figures.

### DETAILED DESCRIPTION

The exemplary embodiments of the spinal implant system and method are discussed in terms of medical devices for the treatment of musculoskeletal disorders and more particularly, in terms of a surgical system and method for treatment of a spine disorder. In some embodiments, the spinal implant system and method may be employed in applications such as correction of deformities, such as, for example, scoliosis. For example, the spinal implant system and method can include attachment of a tether to a first side, such as, for example, a convex side of a spine that is curved due to scoliosis. In some embodiments, while the tether may be affixed to a first side of each of a plurality of vertebrae to prevent growth of vertebrae of the first side, the system allows for growth and adjustments to a second side, such as, for example, a concave side of the plurality of vertebrae. In one embodiment, the spinal implant system and method provides for resistance to and/or prevention of the backing out of a bone fastener, for example, a screw, from an opening of the spinal implant caused by the growth and adjustments of the plurality of vertebrae.

In one embodiment, the system includes a plate that provides an anti-backout feature for a screw within the plate. In one embodiment, the anti-backout feature has a spring and lock mechanism to contain the spring within the plate. In one embodiment, the system includes an anti-backout mechanism provided on a double plate, which can include at least two openings for fasteners. In one embodiment, the system is employed with a fusionless deformity correction tether system placed anteriorly with vertebrae. In one embodiment, the system includes a spring that is deflected during screw insertion and returns back to its original position after a screw shoulder passes the spring. In one embodiment, the system includes a stop for engaging the spring, which is press fit or rigidly inserted into the plate after the spring is assembled into the plate. The stop is configured to resist and/or prevent the spring from exiting the plate forward or backward. In one embodiment, the stop resists and/or prevents the spring from compressing or closing to resist the spring from protruding and/or slipping out of the screw side of the plate. In one embodiment, the system includes a plate having a housing mechanism that supports a spring. In one embodiment, the housing is separate from and integrally connected to the plate. In one embodiment, the housing is threaded with a plate such that the spring and housing are selectively rotated to a selected angular orientation, such as, for example, 90 degrees, to allow the screw to be removed from the plate. It is envisioned that the plate includes various capturing mechanisms to support the housing and/or spring. In one embodiment, the housing is threaded into the plate and the housing is frictionally held or pressure fit with the plate for stability and movement between each position requiring actuation using a driving mechanism.

In some embodiments, one or all of the components of the spinal implant system may be disposable, peel-pack, pre-packed sterile devices. One or all of the components of the spinal implant system may be reusable. The spinal implant system may be configured as a kit with multiple sized and configured components.

In some embodiments, the present disclosure may be employed to treat spinal disorders, such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed spinal implant system and method may be employed in a surgical treatment with a patient in a prone or supine position, and/or employ various surgical approaches to the spine, including anterior, posterior, posterior mid-line, direct lateral, postero-lateral, and/or antero-lateral approaches, and in other body regions. The present disclosure may also be employed with procedures for treating the lumbar, cervical, thoracic and pelvic regions of a spinal column. The present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

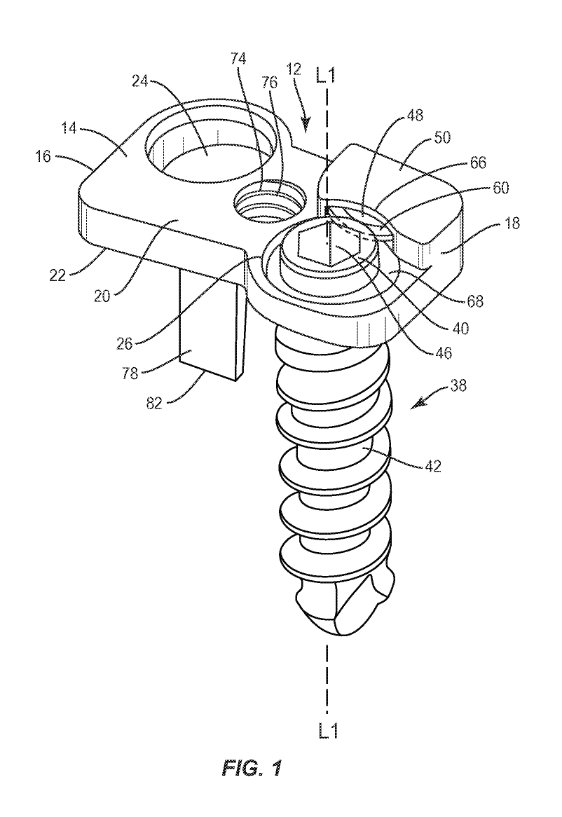

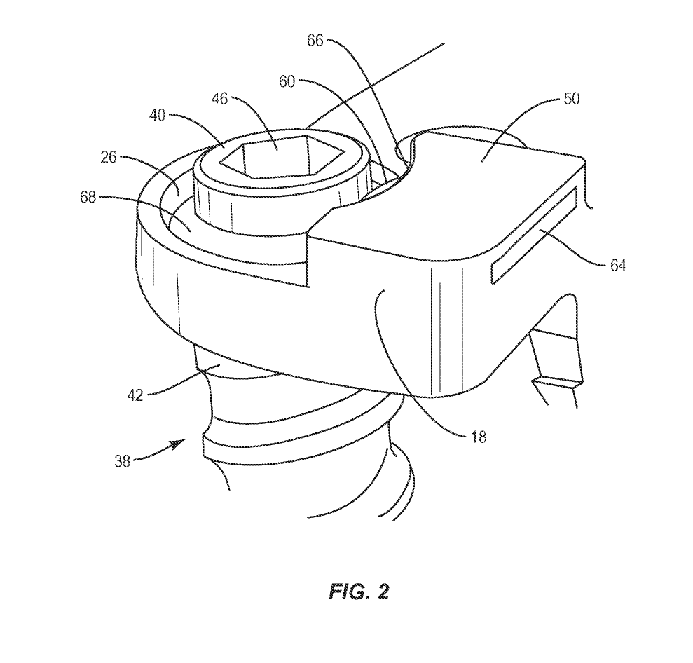

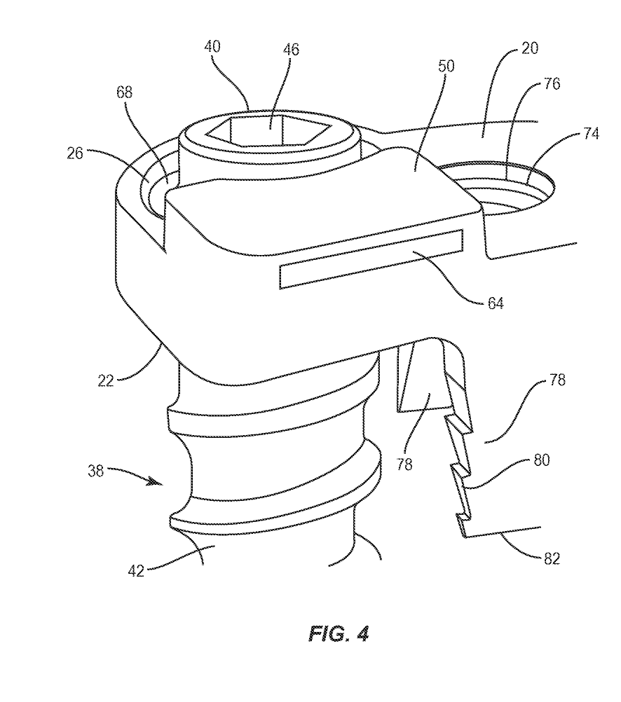

The following discussion includes a description of a spinal implant system, related components and methods for employing the spinal implant system. Alternate embodiments are also disclosed. Reference will now be made in detail to the exemplary embodiments of the present disclosure, which are illustrated in the accompanying figures. Turning now to FIGS. 1-6, there is illustrated components of a spinal implant system, such as, for example, a spinal correction system 10 in accordance with the principles of the present disclosure.

The components of system 10 can be fabricated from biologically acceptable materials suitable for medical applications, including metals, synthetic polymers, ceramics and bone material and/or their composites, depending on the particular application and/or preference of a medical practitioner. For example, the components of system 10, individually or collectively, can be fabricated from materials such as stainless steel alloys, commercially pure titanium, titanium alloys, Grade 5 titanium, super-elastic titanium alloys, cobalt-chrome alloys, stainless steel alloys, super elastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of system 10 may have material composites, including the above materials, to achieve various desired characteristics such as strength, rigidity, elasticity, compliance, biomechanical performance, durability and radiolucency or imaging preference. The components of system 10, individually or collectively, may also be fabricated from a heterogeneous material such as a combination of two or more of the above-described materials. The components of system 10 may be monolithically formed, integrally connected or include fastening elements and/or instruments, as described herein.

System 10 is employed, for example, with an open, mini-open or minimally invasive surgical technique to attach a longitudinal element, such as, for example, a tether, to a first side, such as, for example, a convex side of a spine that has a spinal disorder. The tether may be attached to a bone fastener, which is disposed with an implant, such as, for example, a plate, affixed to the convex side of each of a plurality of vertebrae to prevent growth of vertebrae of a selected section of the spine. System 10 allows for growth and adjustments to a second side, such as, for example, a concave side of the plurality of vertebrae for a correction treatment to treat various spine pathologies, such as, for example, adolescent idiopathic scoliosis and Scheuermann's kyphosis.

System 10 includes a spinal implant, such as, for example, a plate 12. Plate 12 has a substantially rectangular configuration. In some embodiments, plate 12 can be variously configured, such as, for example, tubular, oval, oblong, triangular, square, polygonal, irregular, uniform, non-uniform, variable, hollow and/or tapered. Plate 12 includes a wall 14 extending between a first end 16 and a second end 18. Wall 14 has a substantially rectangular cross section and defines a thickness that is substantially non-uniform to accommodate components of system 10. In some embodiments, wall 14 can have alternate cross-section and/or thickness configurations, such as, arcuate, undulating, offset, staggered, tubular, oval, oblong, triangular, square, polygonal, irregular, uniform, variable, hollow and/or tapered.

Wall 14 has a first surface 20 and a second surface 22. Surface 22 includes substantially planar portions and is oriented in a first direction such that all or only a portion of surface 22 faces and/or engages tissue, as will be described. Surface 20 is oriented in a second direction, opposite to the first direction. In one embodiment, surface 22 is oriented in a posterior or postero-lateral direction for engagement with vertebral tissue. In one embodiment, surface 22 has a frictional surface configuration for engagement with tissue to enhance fixation. In some embodiments, surface 22 may include alternate surface configurations, such as, for example, rough, arcuate, undulating, mesh, porous, semi-porous, dimpled and/or textured according to the requirements of a particular application.

Plate 14 has a double plate configuration and includes a first opening 24 and a second opening 26 spaced apart from opening 24 along surface 20. Openings 24, 26 are substantially circular and extend through the thickness of wall 14. In some embodiments, opening 24 and/or opening 26 can be variously configured, such as, for example, oval, oblong, triangular, square, polygonal, irregular, uniform, non-uniform and/or tapered.

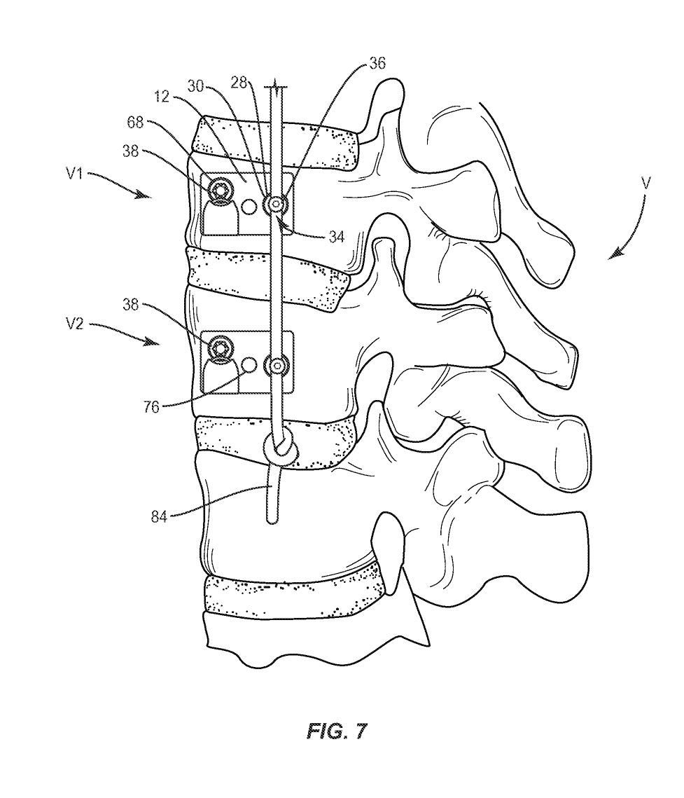

Opening 24 is configured to receive a bone fastener, such as, for example, a bone screw 28 (FIG. 7) that connects a longitudinal element, such as, for example, a tether, to plate 12 and/or tissue, as will be described. Bone screw 28 has a length that is extendable along a longitudinal axis. Bone screw 28 comprises a first portion, such as, for example, a head 30 and a second portion, such as, for example, an elongated shaft (not shown) configured for penetrating tissue.

The shaft of bone screw 28 has a cylindrical cross section configuration and includes an outer surface having an external thread form. In some embodiments, the external thread form may include a single thread turn or a plurality of discrete threads. In some embodiments, other engaging structures may be located on the shaft of bone screw 28, such as, for example, a nail configuration, barbs, expanding elements, raised elements and/or spikes to facilitate engagement of the shaft of bone screw 28 with tissue, such as, for example, vertebrae.

In some embodiments, all or only a portion of the shaft of bone screw 28 may have alternate cross section configurations, such as, for example, oval, oblong, triangular, square, polygonal, irregular, uniform, non-uniform, offset, staggered, undulating, arcuate, variable and/or tapered. In some embodiments, the outer surface of the shaft of bone screw 28 may include one or a plurality of openings. In some embodiments, all or only a portion of the outer surface of the shaft of bone screw 28 may have alternate surface configurations to enhance fixation with tissue such as, for example, rough, arcuate, undulating, mesh, porous, semi-porous, dimpled and/or textured according to the requirements of a particular application. In some embodiments, all or only a portion of the shaft of bone screw 28 may be disposed at alternate orientations, relative to the longitudinal axis, such as, for example, transverse, perpendicular and/or other angular orientations such as acute or obtuse, co-axial and/or may be offset or staggered. It is further envisioned that all or only a portion of the shaft of bone screw 28 may be cannulated.

Head 30 includes a pair of spaced apart arms having an inner surface that defines a U-shaped passageway 34. Passageway 34 is configured for disposal of an implant, such as the longitudinal element. In some embodiments, all or only a portion of passageway 34 may have alternate cross section configurations, such as, for example, oval, oblong, triangular, square, polygonal, irregular, uniform, non-uniform, offset, staggered, and/or tapered. In some embodiments, the arms of head 30 may be disposed at alternate orientations, relative to the longitudinal axis, such as, for example, transverse, perpendicular and/or other angular orientations such as acute or obtuse, co-axial and/or may be offset or staggered.

The inner surface of head 30 includes a thread form configured for engagement with a coupling member, such as, for example, a set screw 36. Set screw 36 is threaded with head 30 to attach, fix and/or lock the longitudinal element with bone screw 28 and/or plate 12, as described.

Opening 26 is configured to receive a bone fastener, such as, for example, a bone screw 38 that connects plate 12 with tissue, as will be described. Bone screw 38 extends along a longitudinal axis L1. Bone screw 38 comprises a first portion, such as, for example, a head 40 and a second portion, such as, for example, an elongated shaft 42 configured for penetrating tissue, similar to the elongated shaft of bone screw 28 described above.

Head 40 includes an inner surface that defines a socket cavity 46 configured for engagement with a tool or instrument for inserting and tensioning bone screw 38 with tissue and/or plate 12. Cavity 46 receives a surface of a drive element of the tool that matingly engages the inner surface of head 40 for manipulating bone screw 38. In some embodiments, the surfaces of cavity 46 and the drive element can be alternatively configured, such as, for example, thread form, triangular, square, polygonal, hexalobular, star, torx, irregular, uniform, non-uniform, offset, staggered and/or tapered.

In some embodiments, system 10 can include one or a plurality of bone fasteners such as those described herein and/or fixation elements, which may be employed with a single vertebral level. In some embodiments, the fixation elements may be engaged with vertebrae in various orientations, such as, for example, series, parallel, offset, staggered and/or alternate vertebral levels. In some embodiments, the bone fasteners and/or fixation elements may include one or a plurality of anchors, tissue penetrating screws, conventional screws, expanding screws, wedges, anchors, buttons, clips, snaps, friction fittings, compressive fittings, expanding rivets, staples, nails, adhesives, posts, fixation plates and/or posts. These bone fasteners and/or fixation elements may be coated with an osteoinductive or osteoconductive material to enhance fixation, and/or include one or a plurality of therapeutic agents.

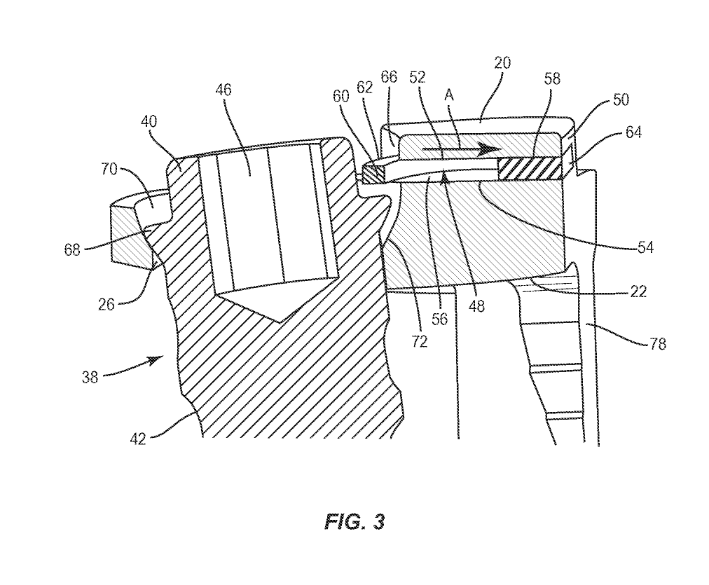

Wall 14 defines a cavity 48 disposed between surfaces 20, 22. Cavity 48 communicates with opening 26. Plate 12 includes a capture member, such as, for example, a housing 50 that comprises a portion 52 of surface 20 and a portion 54 of surface 22 (FIG. 3). Housing 50 encloses and surrounds cavity 48.

Portion 52 has a substantially even configuration and portion 54 has a stepped configuration. Portions 52, 54 are relatively disposed to define a first section 56 and a second section 58 of cavity 48 such that cavity 48 has a continuous and non-interrupted configuration. Section 56 has a uniform thickness and section 58 has a uniform thickness, which is greater than section 56. In some embodiments, section 56 and/or section 58 may be variously configured, such as, for example, those alternatives described herein. In one embodiment, sections 56, 58 may be spaced apart and/or non-continuous. It is envisioned that section 56 and section 58 may have substantially equal thickness, tapered, diverging, converging and/or section 58 having a lesser thickness than section 56.

Housing 50 extends along a portion of wall 14 and protrudes from surface 20 adjacent opening 26. In one embodiment, housing 50 is monolithically formed with plate 12. In one embodiment, housing 50 is separate and integrally connected with wall 14.

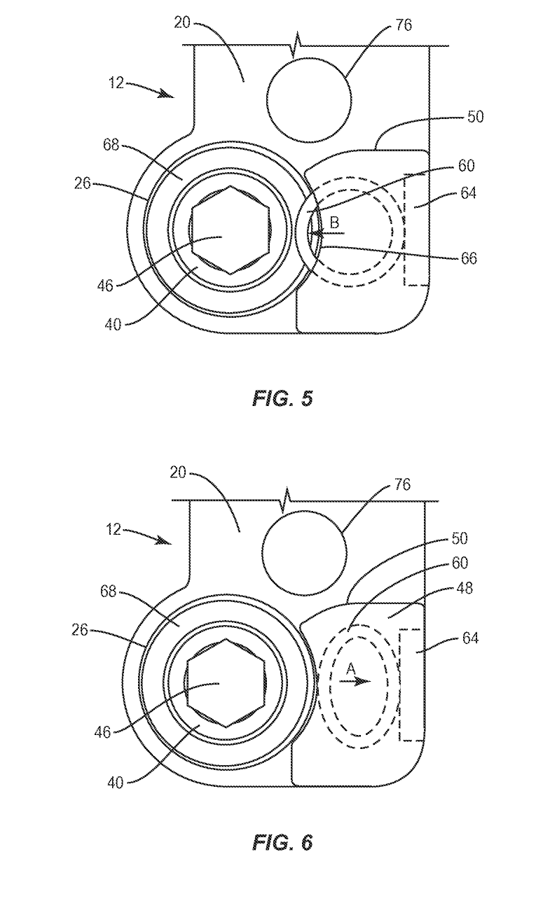

A biasing member, such as, for example, a ring 60 is configured for disposal in section 56 of cavity 48. Ring 60 has a flexible configuration and is engageable with a bone fastener, such as, for example, bone screw 38 to resist backout of bone screw 38 from opening 26. Ring 60 is configured to deflect and/or deform about bone screw 38 between a first, locking orientation, as shown in FIGS. 5 and 6, to resist backout of bone screw 38 from opening 26 and a second, non-locking orientation, as shown in phantom in FIG. 6, such that bone screw 38 is movable through opening 26. In the first orientation, ring 60 is disposed in its naturally biased configuration such that ring 60 is expanded, relative to the second orientation, to resist and/or prevent movement within opening 26. In the second orientation, ring 60 is caused to deflect and at least a portion thereof is collapsed from the biased configuration to permit movement of screw 38 through opening 26.

In one embodiment, ring 60 includes an elastic O-ring that is radially compressible and includes a cylindrical wall 62 having a uniform thickness/diameter. Ring 60 has a resiliently biased configuration such that ring 60 collapses to the second orientation and expands to the first orientation. In some embodiments, all or only a portion of ring 60 may have a semi-rigid, rigid or elastic configuration, and/or have elastic properties, such as the elastic properties corresponding to the material examples described above, such that ring 60 provides a selective amount of expansion, contraction, collapse and/or extension. In some embodiments, ring 60 may be compressible in an axial direction. Ring 60 can include a plurality of separately attachable or connectable portions or sections, such as bands or loops, or may be monolithically formed as a single continuous element.

In some embodiments, ring 60 can have a uniform thickness/diameter. In some embodiments, ring 60 may have various surface configurations, such as, for example, rough, arcuate, undulating, porous, semi-porous, dimpled, polished and/or textured according to the requirements of a particular application. In some embodiments, the thickness/diameter defined by ring 60 may be uniformly increasing or decreasing, or have alternate diameter dimensions along its length. In some embodiments, ring 60 may have various cross section configurations, such as, for example, oval, oblong, triangular, rectangular, square, polygonal, irregular, uniform, non-uniform, variable and/or tapered.

In some embodiments, ring 60 may be braided, such as a rope, or include a plurality arcuate elements to provide a predetermined force resistance. In one embodiment, the biasing member includes an axial element, such as, for example, a flexible rod. In one embodiment, the biasing member has a solid disc shape. In one embodiment, the biasing member has a tubular wall. In some embodiments, the biasing member may include an elastomeric member, clip, leaf spring, gravity induced configuration, pneumatic configuration, hydraulic configuration and/or manual lever.

A stop member 64 is disposed in section 58 of cavity 48 between first and second surfaces 20, 22 and is engageable with ring 60 to resist movement of ring 60. Stop member 64 is press fit or rigidly inserted into plate 12 after ring 60 is assembled into plate 12. Stop member 64 prohibits ring 60 from exiting plate 12 by not allowing ring 60 to exit cavity 48 through section 58.

In operation, a portion of ring 60 protrudes from cavity 48 to overlap opening 26. It is envisioned that ring 60 protrudes from a front portion 66 of housing 50 to overlap opening 26. Ring 60 is disposed in a first orientation such that ring 60 is disposed in its naturally biased configuration prior to introduction of bone screw 38 into opening 26 as shown in FIG. 5. A ledge 72 extends radially into second opening 26. Ledge 72 is configured to provide an axial translation limit of bone screw 38.

Plate 12 has an inner surface 74 that defines a third opening 76 disposed in a central position of wall 14. Opening 76 is spaced apart from openings 24, 26 along surface 20. Opening 76 is substantially circular and extends through the thickness of wall 14. Opening 76 is configured for disposal of a tool or instrument such that the tool is threaded with inner surface 74 for manipulation of plate 12 to introduce and/or deliver plate 12 to a surgical site, as will be described. In some embodiments, plate 12 may include one or a plurality of openings configured for disposal of bone fasteners.

Plate 12 includes a pair of spaced apart transverse extensions 78. Extensions 78 extend substantially perpendicular from surface 22 of plate 12 and are configured to penetrate tissue, such as, for example, bone. Each extension 78 includes fixation elements 80 that are tapered towards its distal end 82. In some embodiments, extensions 78 can have variously configured fixation elements, such as, for example, nails, serrated, textured, staggered, uneven, undulating, smooth, barbs and/or raised elements to facilitate engagement with tissue, such as, for example, a cortical wall of vertebrae.

System 10 includes a flexible longitudinal element, such as, for example, a flexible tether 84, as shown in FIG. 7. Tether 84 has a flexible configuration, which includes movement in a lateral or side to side direction and prevents expanding and/or extension in an axial direction upon fixation with vertebrae. In some embodiments, all or only a portion of tether 84 may have a semi-rigid, rigid or elastic configuration, and/or have elastic properties, such as the elastic properties corresponding to the material examples described above, such that tether 84 provides a selective amount of expansion and/or extension in an axial direction. In some embodiments, tether 84 may be compressible in an axial direction. Tether 84 can include a plurality of separately attachable or connectable portions or sections, such as bands or loops, or may be monolithically formed as a single continuous element. In one embodiment, system 10 includes two tethers. In some embodiments, tether 84 is configured to extend over one or a plurality of vertebral levels.

Tether 84 can have a uniform thickness/diameter. In some embodiments, tether 84 may have various surface configurations, such as, for example, rough, threaded for connection with surgical instruments, arcuate, undulating, porous, semi-porous, dimpled, polished and/or textured according to the requirements of a particular application. In some embodiments, the thickness defined by tether 84 may be uniformly increasing or decreasing, or have alternate diameter dimensions along its length. In some embodiments, tether 84 may have various cross section configurations, such as, for example, oval, oblong, triangular, rectangular, square, polygonal, irregular, uniform, non-uniform, variable and/or tapered.

In some embodiments, tether 84 may have various lengths, according to the requirements of a particular application. In some embodiments, tether 84 may be braided, such as a rope, or include a plurality elongated elements to provide a predetermined force resistance. In some embodiments, tether 84 may be made from autograft and/or allograft, as described above, and be configured for resorbable or degradable applications. In one embodiment, the longitudinal element is a cadaver tendon. In some embodiments, tether 84 may include a cadaver ligament, solid core, tubular element, an artificial strand or a flexible rod.

In assembly, operation and use, a correction system, similar to the system described above, is employed with a surgical procedure, such as, for a correction treatment to treat adolescent idiopathic scoliosis and/or Scheuermann's kyphosis of a spine. In some embodiments, one or all of the components of the spinal correction system can be delivered or implanted as a pre-assembled device or can be assembled in situ. The spinal correction system may be completely or partially revised, removed or replaced.

For example, as shown in FIG. 7, system 10, similar to that described above, can be employed with a surgical correction treatment of an applicable condition or injury of an affected section of a spinal column and adjacent areas within a body, such as, for example, at least a first vertebra V1 and a second vertebra V2, of vertebrae V. In some embodiments, spinal correction system 10 may be employed with one or a plurality of vertebrae.

In use, to treat a selected section of vertebrae V, a medical practitioner obtains access to a surgical site including vertebrae V in any appropriate manner, such as through incision and refraction of tissues. In some embodiments, spinal correction system 10 can be used in any existing surgical method or technique including open surgery, mini-open surgery, minimally invasive surgery and percutaneous surgical implantation, whereby vertebrae V is accessed through a mini-incision, or sleeve that provides a protected passageway to the area. Once access to the surgical site is obtained, the particular surgical procedure can be performed for treating the spine disorder. The configuration and dimension of flexible tether 84 is determined according to the configuration and dimension of the selected section and the requirements of a particular application.

An incision is made in the body of a patient and a cutting instrument (not shown) creates a surgical pathway for implantation of components of system 10. A preparation instrument (not shown) can be employed to prepare tissue surfaces of vertebrae V, as well as for aspiration and irrigation of a surgical region according to the requirements of a particular surgical application.

Pilot holes are made in vertebrae V1, V2 for receiving bone screws 38 that connect plates 12 with vertebrae V1, V2. Plates 12 are removably engaged with a delivery instrument (not shown) via openings 76 and delivered along the surgical pathway. Plates 12 are each delivered to a surgical site adjacent vertebrae V1, V2, respectively. Plates 12 are each oriented for fixation with vertebrae V1, V2, respectively, according to the requirements of a particular application.

Bone screws 38 are delivered along the surgical pathway to the surgical site adjacent vertebrae V1, V2 for penetrating engagement with vertebral tissue. Each bone screw 38 is inserted or otherwise engaged with vertebrae V1, V2, according to the particular requirements of the surgical treatment, to fix each plate 12 with vertebrae V1, V2, respectively.

With plate 12 disposed in a selected orientation relative to a selected vertebra, ring 60 protrudes from cavity 48 to overlap opening 26 in a first orientation, as described herein. As bone screw 38 is axially translated through opening 26, shoulder 68 of bone screw 38 engages ring 60 to overcome the resilient bias of ring 60 such that ring 60 deflects and/or deforms about shoulder 68, in the direction shown by arrow A in FIG. 6. Ring 60 collapses to a second, non-locking orientation, as shown in phantom in FIG. 6, such that bone screw 38 passes along wall 70 of opening 26.

Upon seating of bone screw 38 with wall 70, ring 60 is resiliently biased and expands, in the direction shown by arrow B in FIG. 5, to the first locking orientation to resist and/or prevent movement of bone screw 38 back out of opening 26. Bone screws 38 fix each plate 12 with vertebrae V1, V2, respectively.

Pilot holes are made in vertebrae V1, V2 for receiving bone screws 28 that connect tether 84 to plates 12 and/or vertebrae V1, V2. Bone screws 28 are delivered along the surgical pathway to the surgical site adjacent vertebrae V1, V2 for penetrating engagement with vertebral tissue. Each bone screw 28 is inserted or otherwise engaged with vertebrae V1, V2, according to the particular requirements of the surgical treatment, to fix tether 84 with plates 12 and/or vertebrae V1, V2, respectively

Other components of system 10 are delivered to the surgical site, for example, tether 84 and set screw 36. These components are disposed with bone screws 28, as described above, such that set screw 36 is threaded with head 30, and tether 84 is fixed with bone screw 28 disposed along vertebrae V. Bone screw 28 is configured to support a tensile load with tether 84 over the selected section of vertebrae V.

As shown in FIG. 7, the components of system 10 are attached with a first side, such as, for example, a convex side of vertebrae V to prevent growth of a selected section of vertebrae V, while allowing for growth and adjustments to a second side, such as, for example, a concave side of vertebrae V to provide treatment. Compression of vertebrae V occurs along the convex side. As forces and/or force changes are applied to system 10, such as, for example, patient growth, trauma and degeneration, and/or system 10 component creep, deformation, damage and degeneration, tether 84 adapts with a responsive spring force to maintain the applied force transmitted from bone screw 28 substantially constant.

In one embodiment, system 10 includes an agent, which may be disposed, packed or layered within, on or about the components and/or surfaces of system 10. For example, plate 12 can comprise one or a plurality of surface treatments and/or coatings including the agent. In some embodiments, the agent may include bone growth promoting material, such as, for example, bone graft to enhance fixation of the fixation elements with vertebrae V.

In some embodiments, the agent may include therapeutic polynucleotides or polypeptides. In some embodiments, the agent may include biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as HA, calcium phosphate and calcium sulfite, biologically active agents, for example, gradual release compositions such as by blending in a bioresorbable polymer that releases the biologically active agent or agents in an appropriate time dependent fashion as the polymer degrades within the patient. Suitable biologically active agents include, for example, BMP, Growth and Differentiation Factors proteins (GDF) and cytokines. The components of system 10 can be made of radiolucent materials such as polymers. Radiomarkers may be included for identification under x-ray, fluoroscopy, CT or other imaging techniques. In some embodiments, the agent may include one or a plurality of therapeutic agents and/or pharmacological agents for release, including sustained release, to treat, for example, pain, inflammation and degeneration.

In some embodiments, the use of microsurgical and image guided technologies may be employed to access, view and repair spinal deterioration or damage, with the aid of system 10. Upon completion of the procedure, the surgical instruments, assemblies and non-implant components of system 10 are removed from the surgical site and the incision is closed.

In some embodiments, the components of system 10 may be employed to treat progressive idiopathic scoliosis with or without sagittal deformity in either infantile or juvenile patients, including but not limited to prepubescent children, adolescents from 10-12 years old with continued growth potential, and/or older children whose growth spurt is late or who otherwise retain growth potential. In some embodiments, the components of system 10 and method of use may be used to prevent or minimize curve progression in individuals of various ages.

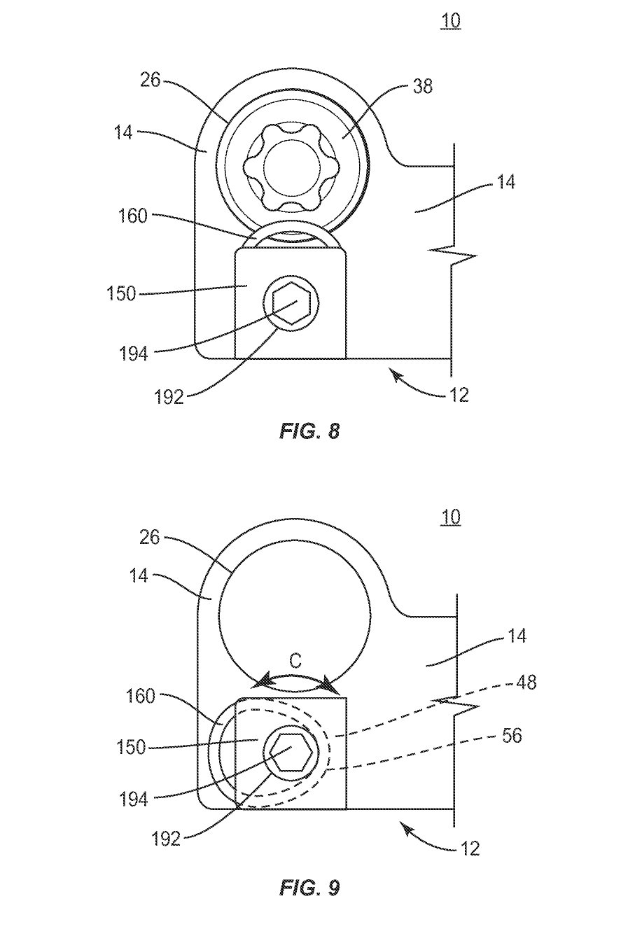

In one embodiment, as shown in FIGS. 8-9, system 10, similar to the systems and methods described with regard to FIGS. 1-7, includes plate 12 having wall 14, described above, and a capture member, such as, for example, a housing 150, similar to housing 50, described above. Wall 14 defines cavity 48 such that housing 150 encloses and surrounds cavity 48, similar to that described above.

A biasing member, such as, for example, a ring 160, similar to ring 60 described above, is configured for disposal in section 56 of cavity 48. Ring 160 has a rigid or semi-rigid configuration and is engageable with bone screw 38 to resist backout of bone screw 38 from opening 26. Ring 60 is rotatable, in the direction shown by arrow C in FIG. 9, about an axis perpendicular to plate 12, between a first, locking orientation, as shown in FIG. 8, to resist backout of bone screw 38 (FIG. 1) from opening 26 and a second, non-locking orientation, as shown in FIG. 9, such that bone screw 38 is movable through opening 26, similar to that described above.

Housing 150 includes a rotatable shroud 192 that is threaded with wall 14 such that shroud 192 and ring 160 are selectively rotated to a selected angular orientation, such as, for example, 90 degrees, to allow bone screw 38 to be removed from plate 12. In one embodiment, shroud 192 is threaded into wall 14 so that shroud 192 is frictionally held with wall 14 for stability and movement between the non-locking and locking orientations.

Shroud 192 includes an inner surface that defines a socket cavity 194 configured for engagement with a tool or instrument for driving the rotation of shroud 192. Cavity 194 receives a surface of a drive element of the tool that matingly engages the inner surface of shroud 192 for manipulating shroud 192 and ring 160. In some embodiments, the surfaces of cavity 194 and the drive element can be alternatively configured, such as, for example, thread form, triangular, square, polygonal, hexalobular, star, torx, irregular, uniform, non-uniform, offset, staggered and/or tapered.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A spinal implant comprising:
a wall having a first surface and a second surface configured to engage tissue, the wall defining a first opening and a second opening configured for disposal of a bone fastener, the wall further defining a cavity disposed between the surfaces and in communication with the second opening; and
a biasing member configured for disposal in the cavity and being engageable with the bone fastener to resist backout of the bone fastener from the second opening.

2. A spinal implant as recited in claim 1, wherein the biasing member is configured to deflect about the bone fastener between a first orientation to resist backout of the bone fastener from the second opening and a second orientation such that the bone fastener is movable through the second opening.

3. A spinal implant as recited in claim 2, wherein the biasing member is resiliently biased to the first orientation.

4. A spinal implant as recited in claim 1, wherein the biasing member includes an elastic ring.

5. A spinal implant as recited in claim 1, wherein the cavity has a uniform thickness between the surfaces.

6. A spinal implant as recited in claim 1, further comprising a stop disposed in the cavity and engageable with the biasing member to resist movement of the biasing member.

7. A spinal implant as recited in claim 6, wherein the cavity has a first thickness configured for disposal of the biasing member and a second, greater thickness configured for disposal of the stop.

8. A spinal implant as recited in claim 1, further comprising a capture member disposed about the biasing member and being configured to move the biasing member into and out of engagement with the bone fastener.

9. A spinal implant as recited in claim 7, wherein the capture member includes a rotatable shroud.

10. A spinal implant as recited in claim 7, wherein the capture member defines a socket engageable with an instrument for rotating the capture member.

11. A spinal implant as recited in claim 1, further comprising a rotatable shroud disposed about the biasing member and configured to move the biasing member between a locking orientation to resist backout of the bone fastener from the second opening and a non-locking orientation such that the bone fastener is movable through the second opening.

12. A spinal implant as recited in claim 1, wherein the wall includes a plate such that the second surface engages anterior tissue.

13. A spinal implant as recited in claim 1, wherein the second surface includes a planar face having at least one transverse extension configured to penetrate tissue.

14. A spinal implant system comprising:
a first bone fastener;
a second bone fastener;
a spinal implant including a wall having a first surface and a second surface configured to engage tissue, the wall defining a first opening configured for disposal of the first bone fastener and a second opening configured for disposal of the second bone fastener, the wall further defining a cavity disposed between the surfaces and in communication with the second opening;
a biasing member configured for disposal in the cavity and engageable with the second bone fastener; and
a longitudinal element attached to the first bone fastener,
wherein the biasing member is configured to deflect about the second bone fastener between a first orientation to resist backout of the second bone fastener from the second opening and a second orientation such that the second bone fastener is movable through the second opening.

15. A spinal implant system as recited in claim 14, wherein the biasing member is resiliently biased to the first orientation.

16. A spinal implant system as recited in claim 14, wherein the cavity has a uniform thickness between the surfaces.

17. A spinal implant system as recited in claim 14, further comprising a stop disposed in the cavity and engageable with the biasing member to resist movement of the biasing member.

18. A spinal implant system as recited in claim 14, further comprising a capture member disposed about the biasing member and being configured to move the biasing member into and out of engagement with the second bone fastener.

19. A spinal implant system as recited in claim 18, wherein the capture member includes a rotatable shroud.

20. A spinal implant system comprising:
a first plate having a first surface and a second surface configured to engage anterior vertebral tissue, the first plate defining a first opening configured for disposal of a first bone fastener and a second opening configured for disposal of a second bone fastener, the first plate further defining a cavity disposed between the surfaces and in communication with the second opening;
a first elastic ring disposed in the cavity and engageable with the second bone fastener;
a second plate having a first surface and a second surface configured to engage anterior vertebral tissue, the second plate defining a first opening configured for disposal of a first bone fastener and a second opening configured for disposal of a second bone fastener, the second plate further defining a cavity disposed between the surfaces and in communication with the second opening of the second plate;
a second elastic ring disposed in the cavity of the second plate and engageable with the second bone fastener; and
a tether attached to the first bone fasteners,
wherein the rings are configured to deflect about a respective second bone fastener between a locking orientation to resist backout of the second bone fastener from a respective second opening and a non-locking orientation such that the second bone fasteners are axially translatable through a respective second opening.
